(19) United States
(12) Patent Application Publication
(10) Pub. No.: US 2022/0315858 A1
(43) Pub. Date: Oct. 6, 2022

(54) AN OIL EXTRACT COMPRISING DIHOMOLINOLENIC ACID, AND A PROCESS FOR PRODUCING THE OIL EXTRACT

(71) Applicant: SEAGREEN BIO LIMITED, Barefield, County Clare (IE)

(72) Inventors: James Joseph LEAHY, Lisnagry (IE); Susan Claire KEATING, Barefield (IE)

(73) Assignee: SEAGREEN BIO LIMITED, Barefield, County Clare (IE)

(21) Appl. No.: 17/595,736

(22) PCT Filed: May 22, 2020
(86) PCT No.: PCT/EP2020/064308

§ 371 Date: Nov. 23, 2021

(30) Foreign Application Priority Data
May 24, 2019 (IE) S2019/0080
Mar. 4, 2020 (IE) 2020/0041

(51) Int. Cl. C11B 1/10; B01D 11/02; B01D 3/10; C11B 3/12

(52) U.S. Cl. CPC C11B 1/10; B01D 11/0288; B01D 11/0296; B01D 3/10; B01D 11/0219; C11B 3/12

(57) ABSTRACT

A solvent extraction process for extracting oil containing dihomolinolenic acid from marine macro-algae, since as ascophyllum. Harvested ascophyllum is desalted to a salt content of less than 3% by weight, and dried to a moisture content of less than 5% by weight, and is then chopped into pieces of maximum dimension not exceeding 5 mm. The desalted, dried and chopped ascophyllum is then packed into a cellulose soxhlet thimble (13) and covered with a silica glass wool. The soxhlet thimble (13) is then placed in a vessel (12) of a soxhlet apparatus (11). A solvent reservoir (15) is charged with the solvent, namely, a food grade hexane, and is evaporated from the solvent reservoir (15) and condensed in a condenser (22) above the vessel (12) and is drip-fed into the soxhlet thimble (13) for extracting the oil containing the dihomolinolenic acid from the ascophyllum. The solvent with the extracted oil entrained therein is returned to the solvent reservoir (15) and the process continues until substantially all the oil has been extracted from the ascophyllum. The oil containing the dihomolinolenic acid is then recovered from the solvent by low pressure distillation until the solvent content of the oil containing the dihomolinolenic acid has been reduced to less than 5% by weight. The oil containing the dihomolinolenic acid is then desolvated to remove the remaining solvent therefrom.

The present invention relates to a process for producing an oil extract comprising dihomolinolenic acid, and the invention also relates to an oil extract comprising dihomolinolenic acid produced by the process.

Dihomolinolenic acid (DGLA) is an extremely uncommon fatty acid which has known anti-inflammatory effects. It is known that marine macro-algae, commonly referred to as seaweed, and in particular, intertidal marine macro-algae contains dihomolinolenic acid. However, known extraction processes for extracting oils from marine macro-algae are capable of producing such extracted oils with only minimal levels of dihomolinolenic acid, and in general, at levels of dihomolinolenic acid of no more than 5% by weight of the extracted oil.

The present invention is directed towards a process for producing an oil extract from marine macro-algae with improved levels of dihomolinolenic acid, and the invention is also directed towards an oil extract extracted from marine macro-algae comprising an improved level of dihomolinolenic acid produced by the process.

According to the invention there is provided a process for producing an oil extract from marine macro-algae, wherein the oil extract comprises dihomolinolenic acid, the process comprising extracting the oil from the marine macro-algae by solvent extraction, the solvent comprising one of an aliphatic hydrocarbon solvent from a C6 aliphatic hydrocarbon solvent to a C17 aliphatic hydrocarbon solvent.

Preferably, the aliphatic hydrocarbon solvent comprises an aliphatic hydrocarbon solvent from a C6 aliphatic hydrocarbon solvent to a C15 aliphatic hydrocarbon solvent, and advantageously, up to a C12 aliphatic hydrocarbon solvent, and preferably up to a C10 aliphatic hydrocarbon solvent.

In one embodiment of the invention, the solvent comprises a food grade solvent.

In one embodiment of the invention the solvent comprises hexane.

In another embodiment of the invention the dihomolinolenic acid constitutes not less than 7% by weight of the oil extract.

Preferably, the oil extract constitutes dihomolinolenic acid in an amount not less than 8% by weight of the oil extract.

Advantageously, the oil extract constitutes dihomolinolenic acid in an amount not less than 10% by weight of the oil extract, and preferably, not less than 11% by weight of the oil extract, and ideally, not less than 12% by weight of the oil extract.

In one aspect of the invention the dihomolinolenic acid constitutes in the range of 7% to 15% by weight of the oil extract.

In another aspect of the invention the dihomolinolenic acid constitutes in the range of 8% to 15% by weight of the oil extract.

In a further aspect of the invention the dihomolinolenic acid constitutes in the range of 10% to 15% by weight of the oil extract, and preferably, in the range of 11% to 15% by weight of the oil extract.

In another embodiment of the invention the marine macro-algae comprises an intertidal marine macro-algae, and preferably, the marine macro-algae comprises marine macro-algae commonly found in the north east Atlantic Ocean.

In one embodiment of the invention the marine macro-algae comprises one or more of the following species: ascophyllum; Fucus vesiculosus; pelvetia canaliculata; cystoseira tamariscifolia; cystoseira nodicaulis; porphyra dioica.

Preferably, the marine macro-algae comprises ascophyllum.

In another embodiment of the invention the marine macro-algae is placed in a first vessel, and the solvent is delivered into the first vessel to extract the oil from the marine macro-algae.

In one embodiment of the invention the solvent is continuously delivered to the first vessel.

In another embodiment of the invention the solvent with the oil extract entrained therein is continuously delivered from the first vessel.

Alternatively, the solvent is drip-fed into the first vessel.

In one embodiment of the invention the first vessel comprises a pressurised vessel.

Preferably, the solvent extraction is carried out in the first vessel under pressure.

Advantageously, the solvent extraction is carried out in the first vessel at a pressure in the range of 10 bar to 50 bar absolute pressure. Ideally, the solvent extraction is carried out in the first vessel at a pressure of approximately 40 bar absolute pressure.

In one embodiment of the invention the solvent is supplied to the first vessel from a second vessel.

In an alternative embodiment of the invention the first vessel comprises a process vessel of a soxhlet apparatus and the second vessel comprises a solvent reservoir attached to the soxhlet apparatus for storing the solvent and receiving the solvent with the oil extract entrained therein from the process vessel.

In one embodiment of the invention the marine macro-algae is covered in the first vessel with a silica glass wool.

In another embodiment of the invention the marine macro-algae is packed into the first vessel.

In another embodiment of the invention the solvent with the oil extract entrained therein is returned from the first vessel to the second vessel.

In one embodiment of the invention the solvent in the second vessel is evaporated and the solvent evaporate is delivered to the first vessel.

In another embodiment of the invention the second vessel is heated to raise the temperature of the contents therein to the boiling point of the solvent.

In another embodiment of the invention the solvent evaporate is condensed prior to delivery to the first vessel.

Preferably, the marine macro-algae is retained in a porous container in the first vessel.

In one embodiment of the invention the porous container comprises a mesh container. Alternatively, the porous container comprises a cellulose container.

In another embodiment of the invention the oil extraction process is carried out in the first vessel at room temperature.

Preferably, the oil extraction process is continued until the solvent being delivered from the first vessel contains substantially no oil.

Advantageously, the oil extraction process is continued until substantially all the oil being extracted from the marine macro-algae has been extracted therefrom. Preferably, substantially all the oil being extracted from the marine macro-algae is determined as having been extracted therefrom by inspecting the solvent being delivered from the first vessel.

Advantageously, substantially all the oil being extracted from the marine macro-algae is determined as having been extracted from the marine macro-algae by inspecting the colour of the solvent being delivered from the first the vessel.

In one embodiment of the invention the oil extraction process is continued until the solvent being delivered from the first vessel is colourless.

In one embodiment of the invention the oil extract entrained in the solvent is recovered from the solvent by distillation.

Preferably, solvent evaporated during distillation is condensed for subsequent use.

In one embodiment of the invention distillation is carried out at low pressure. Preferably, distillation is carried out at a pressure in the range of 10 mbar to 100 mbar absolute pressure. Advantageously, distillation is carried out at a pressure in the range of 20 mbar to 30 mbar absolute pressure. Ideally, the distillation is carried out at a pressure of approximately 20 mbar absolute pressure.

In one embodiment of the invention the distillation is carried out at a temperature of the boiling point of the solvent corresponding to the pressure at which the distillation is being carried out.

Preferably, the distillation is carried out until the solvent contained in the oil extract does not constitute more than 5% by weight of the recovered oil extract.

In one embodiment of the invention the solvent with the oil extract entrained therein is cooled prior to distillation. Preferably, the solvent with the oil extract entrained therein is cooled to a temperature in the range of 20° C. to 30° C. prior to distillation. Advantageously, the solvent with the oil extract entrained therein is cooled to a temperature of approximately of 25° C. prior to distillation.

In another embodiment of the invention the oil extract recovered from distillation is subjected to desolvating until substantially all the solvent has been removed from the recovered oil extract or the amount of solvent remaining in the recovered oil extract has been reduced to a negligible amount. Preferably, the desolvating of the recovered oil extract from distillation is carried out at a temperature in the range of 30° C. to 50° C. Advantageously, the desolvating of the recovered oil extract from distillation is carried out at a temperature in the range of 40° C. to 45° C. Ideally, the desolvating of the recovered oil extract from distillation is carried out at a temperature of approximately 41° C.

In one embodiment of the invention the desolvating of the recovered oil extract from distillation is carried out in a vacuum. Preferably, the desolvating of the recovered oil extract from distillation is carried out at a pressure in the range of 10 mbar to 50 mbar absolute pressure. Advantageously, the desolvating of the recovered oil extract from distillation is carried out at a pressure in the range of 20 mbar to 30 mbar absolute pressure. Ideally, the desolvating of the recovered oil extract from distillation is carried out at a pressure of approximately 20 mbar absolute pressure.

Preferably, the recovered oil extract from distillation is cooled prior to the desolvating thereof. Preferably, the recovered oil extract from distillation is cooled to a temperature in the range of 20° C. to 30° C. prior to desolvating thereof. Advantageously, the recovered oil extract from distillation is cooled to a temperature of approximately 25° C. prior to desolvating thereof.

In one embodiment of the invention the marine macro-algae is desalted prior to the solvent extraction process. Preferably, the marine macro-algae is desalted until the residual salt in the marine macro-algae does not exceed 7% by weight. Advantageously, the marine macro-algae is desalted until the residual salt in the marine macro-algae does not exceed 5% by weight. Ideally, the marine macro-algae is desalted until the residual salt in the marine macro-algae does not exceed 3% by weight.

In another embodiment of the invention the marine macro-algae is dried prior to the solvent extraction process. Preferably, the marine macro-algae is dried until the moisture content thereof is less than 10% by weight. Advantageously, the marine macro-algae is dried until the moisture content thereof is less than 8% by weight. More preferably, the marine macro-algae is dried until the moisture content thereof is less than 5% by weight. Ideally, the marine macro-algae is dried until the moisture content thereof is less than 3% by weight.

In one embodiment of the invention the marine macro-algae is dried subsequent to being desalted.

In one embodiment of the invention the marine macro-algae is chopped into pieces prior to the solvent extraction process. Preferably, the marine macro-algae is chopped into pieces of size, the maximum dimension of each piece not exceeding 10 mm. Advantageously, the marine macro-algae is chopped into pieces of size, the maximum dimension of each piece lying in the range of 100 microns to 10 mm. Preferably, the marine macro-algae is chopped into pieces of size, the maximum dimension of each piece not exceeding 8 mm. Advantageously, the marine macro-algae is chopped into pieces of size, the maximum dimension of each piece not exceeding 5 mm. Ideally, the marine macro-algae is chopped into pieces of size, the maximum dimension of each piece lying approximately 4 mm.

In one embodiment of the invention the marine macro-algae is chopped into pieces subsequent to being dried.

In one embodiment of the invention the oil extract is microencapsulated.

The invention also provides an oil extract extracted from marine macro-algae by the process according to the invention, the oil extract comprising dihomolinolenic acid.

The advantages of the invention are many. A particularly important advantage of the invention is that it provides a process which enables dihomolinolenic acid to be extracted from marine macro-algae at significantly higher yields than can be achieved by known prior art processes. In examples of the process according to the invention, oil extract which is extracted from ascophyllum has been found to contain dihomolinolenic acid in amounts of the order of 12.62% by weight of the extracted oil, 11.48% by weight of the extracted oil and 11.2% by weight of the extracted oil, thus giving an average yield of dihomolinolenic acid of 11.77% by weight of the oil extract. A further advantage of the invention is that the oil extract produced from the ascophyllum according to the process according to the invention, also results in relatively high yields of Palmitic acid, Oleic acid and moderate amounts of Arachidonic, Linolenic and Linoleic fatty acids.

The process according to the invention also provides an efficient process for extracting oil with a relatively high content of dihomolinolenic acid from marine macro-algae, with minimum, and in general, virtually no degradation of the extracted oil, and in particular, no degradation of the linolinolenic acid.

The invention will be more clearly understood from the following description of some preferred embodiments thereof which are given by way of example only with reference to the accompanying drawings, in which:

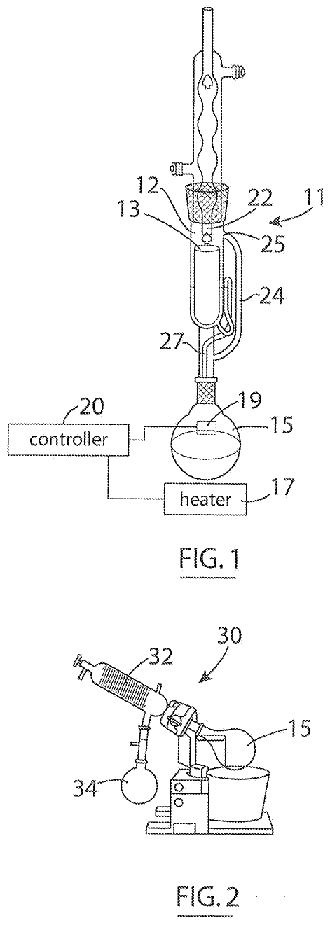

FIG. 1 illustrates a soxhlet apparatus for carrying out a process according to the invention on a pilot scale for extracting an oil extract also according to the invention containing dihomolinolenic acid from marine macro-algae,

FIG. 2 illustrates a low temperature distillation apparatus also for use in the process according to the invention for extracting the oil extract according to the invention from the marine macro-algae,

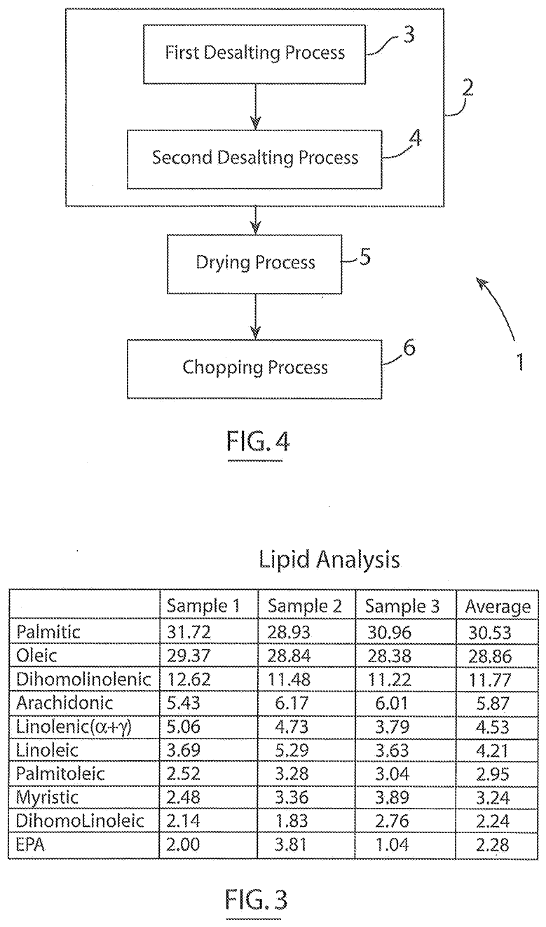

FIG. 3 is a table showing a lipid analysis of the oil extract according to the invention extracted from the marine macro-algae during three repetitions of the process according to the invention as will be described below,

FIG. 4 illustrates a block representation of a prepping process for preparing marine macro-algae of the process according to the invention for extracting the oil extract therefrom, and

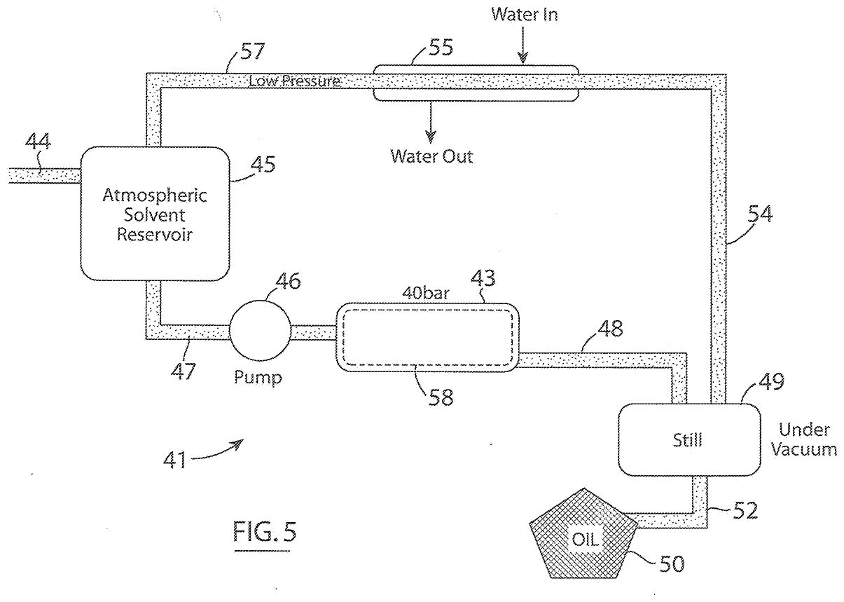

FIG. 5 illustrates a block representation of process apparatus for carrying out the process according to the invention on a commercial scale for producing the oil extract according to the invention.

Referring to the drawings, and initially to FIGS. 1 to 4 thereof, a solvent extraction process according to the invention for extracting an oil extract also according to the invention containing dihomolinolenic acid from marine macro-algae will now be described. In this embodiment of the invention the marine macro-algae comprises an intertidal marine macro-algae, namely, ascophyllum which is harvested from the North Atlantic Ocean, and in this case from the Irish shoreline. However, it will be readily apparent to those skilled in the art that the marine macro-algae may be selected from any one or more of the following marine macro-algae: ascophyllum; Fucus vesiculosus; pelvetia canaliculata; cystoseira tamariscifolia; cystoseira nodicaulis; porphyra dioica, and any other suitable marine macro-algae. It will also of course be appreciated that the marine macro-algae may be harvested from any other suitable ocean or sea.

The harvested marine macro-algae is initially subjected to a prepping process, a block representation of which is illustrated in FIG. 4 and indicated generally by the reference numeral 1, in order to reduce the salt content thereof to 3% by weight or less and the moisture content thereof to less than 5% by weight. The prepping process 1 comprises a first part which comprises a desalting process represented by block 2 of FIG. 4. The desalting process comprises a first desalting step represented by block 3 and a second desalting step represented by block 4 of FIG. 4. The harvested marine macro-algae is subjected to the first and second desalting steps 3 and 4 sequentially.

In the first and second desalting steps 3 and 4, the harvested marine macro-algae is subjected to soaking in fresh water at ambient temperature in first and second baths, respectively. The harvested marine macro-algae is sequentially immersed in the respective first and second baths of fresh water for respective first and second time periods each of approximately 120 minutes. While immersed in the first bath during the first time period of the first desalting step 3, the marine macro-algae is agitated by periodically pressing it down into the fresh water in the first bath, to ensure that it is fully immersed in the fresh water therein. At the end of the first time period of 120 minutes, the marine macro-algae is transferred from the first bath into the second bath and is immersed in fresh water in the second bath for the second time period of 120 minutes of the second desalting step 4, during which time the marine macro-algae is agitated periodically by pressing it down into the fresh water. At the end of the second time period of 120 minutes of the second desalting step 4 the marine macro-algae is removed from the second bath. At that stage the salt content of the marine macro-algae should not exceed 3% by weight. The baths are recharged with fresh water prior to each soak of the marine macro-algae therein.

While it is desirable that the marine macro-algae should be subjected to two soaks in fresh water during the desalting step, in some embodiments of the invention a single soak in fresh water may be sufficient to reduce the salt content of the marine macro-algae to a desired level, and preferably, to a desired level not exceeding 3% by weight. In other embodiments of the invention, it is envisaged that the marine macro-algae may be subjected to more than two soaks in fresh water, and in other embodiments of the invention, it is envisaged that the time duration of each soak may be greater than or less than 120 minutes, and further, it is envisaged that the soak times for each soak may be different from soak to soak. In other embodiments of the invention the desalting process may be carried out by passing fresh water continuously over the marine macro-algae. It will be well understood by those skilled in the art that other suitable processes for desalting marine macro-algae may be used.

On completion of the desalting process 2 of the marine macro-algae, the desalted marine macro-algae is then subjected to a second part of the prepping process which is a drying process represented by block 5 of FIG. 4. During the drying process 5, the marine macro-algae is suspended from racks, and a stream of warm dehumidified air at a temperature in the range of 25° C. to 40° C. is passed over the suspended marine macro-algae. The drying process continues until the moisture content of the dried marine macro-algae in this example has been reduced to less than 5% by weight.

Other suitable drying processes, which will be well known and understood by those skilled in the art may be used for drying the marine macro-algae in order to reduce the moisture content thereof, which preferably, should be reduced to less than 5% by weight. For example, it is envisaged that the marine macro-algae may be dried by passing warm dehumidified air at temperatures other than in the range of 25° C. to 40° C., and in some cases, the air temperature may be less than 25° C., and in other cases the air temperature may be greater than 40° C. The time period for drying the marine macro-algae is not critical, provided that the marine macro-algae is dried to a moisture content of less than 5% by weight, although in some cases, a moisture content higher than 5% by weight may be acceptable.

The desalted and dried marine macro-algae is then subjected to a chopping process represented by block 6 of FIG. 4. The chopping process is carried out in a multi-bladed drum chopper, and the drum chopper is configured to chop the desalted, dried marine macro-algae into pieces of size, the maximum dimension of which is dependent on the scale of the process apparatus in which the oil extraction process is to be carried out. The chopping of the marine macro-algae is carried out in order to improve the efficiency of the extraction of the oil from the marine macro-algae. In this embodiment of the invention the desalted, dried marine macro-algae is chopped into pieces, the maximum dimension of which does not exceed 5 mm, although depending on the process apparatus in which the solvent extraction process is being carried out, the chopped pieces may be of maximum dimensions greater than or less than 5 mm. In general, the maximum dimension of the chopped pieces of the marine macro-algae will be dependent very much on the process apparatus in which the process according to the invention for extracting the oil extract is being carried out, and also on the process according to the invention by which the oil extract is being extracted from the marine macro-algae.

The desalted, dried and chopped marine macro-algae is now ready for the extraction of the oil containing the dihomolinolenic acid therefrom.

Referring now to FIGS. 1 and 2, the extraction of the oil extract containing the dihomolinolenic acid from the marine macro-algae will now be described. In this example, the oil extraction process is carried out on a pilot scale by solvent extraction in apparatus known as a soxhlet apparatus, in this case a 1.2 litre soxhlet apparatus indicated generally by the reference numeral 11. Before describing the process according to this embodiment of the invention, the soxhlet apparatus 11 will first be described.

The soxhlet apparatus 11 comprises a first vessel 12 open to atmosphere, namely, a process vessel 12 of capacity 1.2 litres and of dimensions 645 mm by 57 mm. The desalted, dried and chopped marine macro-algae is contained in a porous container in the process vessel 12, in this case a soxhlet thimble 13 of cellulose material. The desalted, dried and chopped pieces of the marine macro-algae are placed in the soxhlet thimble 13, which is then placed in the process vessel 12. A second vessel, in this embodiment of the invention, a solvent reservoir 15 comprising a three-necked round bottom flask is fitted to the soxhlet apparatus 11, and stores a solvent for delivery to the soxhlet thimble 13 for extracting the oil extract from the marine macro-algae as will also be described below, and also for storing the solvent with the oil extract entrained therein during the extraction process.

A heating system comprising an electrically powered heater 17 illustrated in block representation in FIG. 2 is provided for heating the solvent in the solvent reservoir 15 for evaporating the solvent therein to provide a solvent evaporate. The solvent evaporate is delivered to a water cooled condenser 22 in the process vessel 12 where it is condensed and drip fed to the soxhlet thimble 13. A temperature sensing means, in this embodiment of the invention comprising a thermocouple 19 located on the solvent reservoir 15 produces a signal indicative of the temperature of the contents of the solvent reservoir 15.

A control means for controlling the temperature of the contents of the solvent reservoir 15 comprises a controller 20 under the control of which the heater 17 is operated. The controller 20 which is illustrated in block representation in FIG. 2 may be any suitable controller, for example, a programmable logic controller, or other suitable signal processor. The controller 20 reads the signal from the thermocouple 19 and operates the heater 17 to maintain the temperature of the contents of the solvent reservoir 15 at a temperature substantially equal to the boiling point temperature of the solvent at atmospheric pressure, in this case at a temperature of approximately 70° C. Alternatively, the temperature sensing means may comprise a thermostatically operated switch, for example, a bimetal switch through which an electrical power supply would be applied to the heater 17. It is also envisaged that other suitable heating means for heating the solvent in the solvent reservoir 15 may be provided. For example, it is envisaged that the heating means may comprise a gas powered heater or other suitable heating means.

An outer tube 24 of the soxhlet apparatus 11 accommodates the solvent evaporate from the solvent reservoir 15 to the process vessel 12 through an inlet port 25, where the solvent evaporate is delivered into the process vessel 12 to impinge upon the condenser 22 for condensing thereof. The condensed solvent drip-feeds from the condenser 22 into the soxhlet thimble 13. A return tube 27 from the process vessel 12 returns the solvent with the oil extract extracted from the marine macro-algae entrained therein to the solvent reservoir 15.

In the process according to this embodiment of the invention 600 gms of the desalted dried and chopped marine macro-algae is placed and packed into the soxhlet thimble 13 and is covered with silica glass wool. The soxhlet thimble 13 with the chopped marine macro-algae therein is placed in the process vessel 12.

The solvent reservoir 15 is charged with 2.8 litres of solvent, which in this embodiment of the invention comprises a food grade hexane with a boiling point of approximately 70° C. The solvent reservoir 15 is then connected to the soxhlet apparatus 11 by the outer tube 24 and the return tube 27 communicating the solvent reservoir 15 with the process vessel 12. Since the solvent in this embodiment of the invention is food grade hexane with a boiling point of approximately 70° C., the controller 20 is set to operate the heater 17 to maintain the temperature of the contents of the solvent reservoir 15 at a temperature of approximately 70° C. The solvent evaporate is delivered from the solvent reservoir 15 to the process vessel 12 through the outer tube 24 and is condensed in the process vessel 12 above the soxhlet thimble 13 by the condenser 22. The condensed solvent is in turn continuously drip-fed from the condenser 22 into the soxhlet thimble 13 to the marine macro-algae therein.

The solvent commences to extract the oil from the marine macro-algae in the soxhlet thimble 13, and solvent entrained with the oil extract passes through the soxhlet thimble 13 into the process vessel 12, and is returned through the return tube 27 to the solvent reservoir 15. This process continues with the solvent with the oil extract entrained therein being returned to the solvent reservoir 15 from the process vessel 12, and the solvent in turn being evaporated in the solvent reservoir 15, and the solvent evaporate being returned to the process vessel 12 until substantially all the oil has been extracted from the marine macro-algae, in other words until the solvent being returned from the process vessel 12 to the solvent reservoir 15 contains no further oil extract. The solvent returning to the solvent reservoir 15 from the process vessel 12 is inspected, and on the returning solvent being detected as being colourless, the solvent is determined as being free of the oil extract, and thus substantially all the oil is determined as having been extracted from the marine macro-algae in the soxhlet thimble 13. Once substantially all the oil is determined as having been extracted from the marine macro-algae, the solvent reservoir 15 is disconnected from the soxhlet apparatus 11 and from the heater 17, and is allowed to cool to a temperature of approximately 25° C.

The extracted oil, which is entrained in the solvent in the solvent reservoir 15, is then recovered by low pressure distillation. A suitable low pressure distillation system is illustrated in FIG. 2 and is indicated generally by the reference numeral 30. The solvent reservoir 15 is coupled to the low pressure distillation system 30. The low pressure distillation is carried out at a pressure of approximately 20 mbar absolute pressure. The solvent reservoir 15 is agitated and heated in the low pressure distillation system 30 to a temperature not exceeding 40° C., which is the approximate boiling point temperature of the solvent at the pressure of approximately 20 mbar absolute pressure, to evaporate the solvent. The solvent evaporate is then condensed in a water cooled condenser 32 and collected in a solvent recovery flask 34 for subsequent use. The low pressure distillation is continued until the amount of solvent remaining in the recovered concentrated oil extract in the solvent reservoir 15 is no more than 5% by weight.

Other suitable low pressure distillation systems besides that illustrated in FIG. 2 may be used in order to reduce the solvent content of the recovered concentrated oil extract to a value of not more than 5% by weight of the solvent. However, it is desirable that the process for reducing the solvent content of the recovered concentrated oil extract to being of a value not more than 5% by weight, should be carried out at a temperature not exceeding 40° C., and if possible, at a lower temperature, in order to minimise any possible degrading of the recovered concentrated oil extract. It will also of course be appreciated that prior to commencing the low pressure distillation for reducing the solvent content of the recovered concentrated oil extract to no more than 5% by weight, the recovered concentred oil extract may be transferred from the solvent reservoir 15 to another suitable container in which the low pressure distillation process would be carried out.

The recovered oil extract from distillation with the 5% solvent content is allowed to cool to a temperature of approximately 25° C., and is then subjected to a desolvating step. The desolvating of the recovered oil extract is carried out at a temperature of approximately 40° C. at a pressure of approximately 20 mbar absolute pressure for approximately 12 hours to remove the remainder of the solvent from the recovered oil extract, or to reduce the solvent content of the recovered oil extract to a negligible amount.

On removal of the solvent reservoir 15 from the soxhlet apparatus 11, the soxhlet thimble 13 with the spent marine macro-algae therein is removed from the process vessel 12. The spent marine macro-algae is disposed of and the soxhlet thimble 13 is dried for reuse. A fresh soxhlet thimble 13 charged with the next batch of marine macro-algae is placed in the process vessel 12, and the removed solvent reservoir 15 is replaced with a fresh solvent reservoir 15 charged with the solvent, and connected to the soxhlet apparatus 11. The solvent extraction process is then repeated on the fresh batch of the marine macro-algae in the soxhlet thimble 13.

It has been found that the recovered concentrated oil extract produced by the process according to the invention carried out in the soxhlet apparatus 11 is rich in dihomolinolenic acid. In fact, it has been found that in three separate repetitions of the above described process, each of which was carried out in accordance with the description above with the marine macro-algae comprising ascophyllum and the solvent being food grade hexane, the oil extract was found to contain an average of 11.77% by weight of dihomolinolenic acid, which is significantly higher than yields achieved by prior art processes.

Referring now to the table of FIG. 4, the percentages of the individual oils contained in the extracted oil from the marine macro-algae in the three repetitions of the process are set forth. As can be seen in the first example, the oil extract contained dihomolinolenic acid in an amount of 12.62% by weight, and in the second example the oil extract contained dihomolinolenic acid in an amount of 11.48% by weight, while in the third sample, the oil extract contained dihomolinolenic acid in an amount of 11.22% by weight, thus giving an average value for the percentage of dihomolinolenic acid in the oil extract in the three repetitions of the process of 11.77% by weight.

Within the lipid profile of the macroalgal oil, the following relative concentrations were measured: Palmitic acid 30.5% followed by Oleic acid 28.9% and Dihomolinolenic acid 11.8%. Moderate amounts of fatty acids are Arachidonic 5.9%, Linolenic 4.5% and Linoleic 4.2% fatty acids. The least amounts of fatty acids were EPA, Dihomolinoleic, Myristic and Palmitoleic 2% to 4% each. Of these fatty acids, the essential fatty acids are Dihomolinolenic, Arachidonic, Linolenic, Linoleic and EPA.

Palmitic and oleic acid comprise almost 60% of the total content of the oil analysed. Palmitic acid is a common saturated fatty acid and can be found in animals, plants and microorganisms. It can be used as an emollient, surfactant, emulsifier and opacifying agent. It has been found to improve percutaneous absorption and has also been found to have anti-oxidant activity. Oleic acid is an unsaturated fatty acid and can be used as an emulsifier and moisturiser and has been found to have anti-oxidant and anti-inflammatory activity. Oleic acid has also been found to improve percutaneous absorption (Vermaak et al, 2011).

It has been found that the oil extracted according to the process described above from ascophyllum, by solvent extraction with the solvent being food grade hexane, has significant anti-inflammatory effects which it is believed are due to the relatively high concentration of dihomolinolenic acid, and is particularly suitable as an active ingredient for an anti-inflammatory composition for topical application. Furthermore, since the oil extract is extracted by food grade hexane, the oil extract is also suitable as an active ingredient for an anti-inflammatory composition for oral administration. It has also been found that the oil extract has ultraviolet rays blocking capacity, and is therefore also suitable in its concentrated form or as an active ingredient for a sunscreen composition for topical application. It is believed that the oil extract may be suitable for cosmetic applications, and may have hair enhancing properties, and may also be suitable as a health and/or food supplement.

It is envisaged that in order to allow the oil extract to be more easily formulated into useable compositions, and also, to mask any odours of the oil extract, the oil extract in the concentrated form recovered from the solvent may be microencapsulated. Such microencapsulation processes will be understood by those skilled in the art.

Referring now to FIG. 5 a process according to the invention on a commercial scale will now be described for extracting dihomolinolenic acid from marine macro-algae by solvent extraction. FIG. 5 illustrates a commercial scale apparatus indicated generally by the reference numeral 41, in which the process according to the invention for extracting the dihomolinolenic acid from the marine macro-algae is carried out.

The apparatus 41 comprises a first vessel 43 in which the dihomolinolenic acid is extracted from the marine macro-algae by solvent extraction. The first vessel 43 is a pressure vessel, and the extraction of the dihomolinolenic acid from the marine macro-algae is carried out in the first vessel 43 under pressure.

A second vessel 45 stores the solvent, which in this embodiment of the invention comprises a food grade hexane which is stored in the second vessel 45 at atmospheric pressure. A solvent supply conduit 44 supplies the solvent to the second vessel 45.

A pump 46 pumps the solvent from the second vessel 45 through a first delivery conduit 47 to the first vessel 43 at a pressure of approximately 40 bar absolute pressure. The solvent with the oil extract entrained therein is delivered from the first vessel 43 through a second delivery conduit 48 to a low pressure distillation apparatus 49 where the oil extract is separated from the solvent by evaporating the solvent. The oil extract extracted from the solvent in the low pressure distillation apparatus 49 is delivered to a storage vessel 50 through a third delivery conduit 52. Evaporated solvent from the low pressure distillation apparatus 49 is returned through a first return conduit 54 to a water cooled condenser 55, where the solvent evaporate is condensed and returned through a second return conduit 57 to the second vessel 45 for reuse.

Turning now to the process according to the invention carried out in the apparatus 41 for extracting dihomolinolenic acid from marine macro-algae, the marine macro-algae, which in this embodiment of the invention is also ascophyllum is initially desalted as already described to a salt content of preferably, not more than 3% by weight, but may in some circumstances be up to but not more than 7% by weight although preferably should be less than 5% by weight. The desalted marine macro-algae is then dried to a moisture content less than 5%, as already described. The desalted and dried marine macro-algae, is then chopped, as already described, into pieces of maximum dimension not more than 10 mm, but typically, of maximum dimension not exceeding 5 mm. The desalted, dried and chopped marine macro-algae are then placed in a mesh container 58, which is then placed in the first vessel 43. The chopped marine macro-algae is placed in the mesh container 58 in order to retain the chopped pieces of the marine macro-algae therein, so that the chopped marine macro-algae is retained within the first vessel 43. With the mesh container 58 charged with the chopped pieces of marine macro-algae placed in the container 43, the container 43 is then sealed. If the second vessel 45 has not yet been charged with the solvent, the second vessel 45 is charged therewith.

The apparatus 41 is now ready to carry out the solvent extraction process on the marine macro-algae.

The pump 46 is activated to pump the solvent from the second vessel 45 to the first vessel 43 where the pump 46 delivers the solvent to the first vessel 43 at a pressure of approximately 40 bar absolute pressure. The solvent with the oil extract contained therein is delivered through the second delivery conduit 48 to the low pressure distillation apparatus 49. The oil extract is separated from the solvent in the low pressure distillation apparatus 49 at a pressure of approximately 20 mbar absolute pressure and at a temperature of 40° C. The separated oil extract, which should have a residual solvent content of not more than 5% by weight, is then delivered through the third delivery conduit 52 to the storage vessel 50, where it is stored for further processing to remove or reduce the solvent remaining in the oil extract to a negligible amount. The evaporated solvent is returned to the second vessel 45 through the condenser 55. The flow rate at which the solvent is passed through the first vessel 43 will be dependent on the volume of the first vessel 43, and will be at a rate to achieve solvent extraction of the oil extract efficiently.

The solvent supply conduit 44 supplies the solvent to the second vessel 45, and the second vessel 45 is topped-up through the solvent supply conduit during the solvent extraction process should the level of solvent in the second vessel 45 drop below a predefined minimum level.

The process is a continuous process, and is continued until substantially all the oil in the marine macro-algae has been extracted to a level below which further solvent extraction become inefficient. Typically, the solvent being delivered from the first vessel 43 to the low pressure distillation apparatus 49 is visually monitored, and when it is visually apparent that the level of oil contained in the solvent is at a level that further solvent extraction would become inefficient, the process is terminated.

On termination of the process, the first vessel 43 is depressurised and opened. The mesh container 58 with the spent, chopped marine macro-algae contained therein is then removed from the first vessel 43, and is replaced with a replacement mesh container 58 charged with a fresh batch of desalted, dried and chopped marine macro-algae, and the process is repeated with the fresh batch of marine macro-algae.

At any stage during the process, or after a number of processes, or at the end of each process the oil extract is delivered from the storage vessel 50 for further processing to remove any residual solvent remaining in the oil extract or to reduce the solvent remaining therein to a negligible amount. This part of the process requires subjecting the oil extract to a desolvating process which is similar to that already described.

While the marine macro-algae, from which the oil extract has been produced in the above described processes, has been described as comprising ascophyllum, it is believed that oil extracts which contain substantially similar amounts of dihomolinolenic acid to those described above may also be solvent extracted using the processes according to the invention as described above from the following marine macro-algae or a mixture thereof: ascophyllum; Fucus vesiculosus; pelvetia canaliculata; cystoseira tamariscifolia; cystoseira nodicaulis; porphyra dioica.

While the solvent has been described as being food grade hexane, while this is desirable in cases where the oil extract is to form an active ingredient for a composition to be orally administered, in cases where the oil extract is required as an active ingredient for a composition for topical application, non-food grade hexane may be used. It is believed that instead of the solvent extraction process being carried out with hexane or food grade hexane, as the solvent, the solvent extraction may be carried out with other long chain aliphatic hydrocarbon solvents. In fact, it is believed that the solvent extraction process could be carried out with any one or more of long chain aliphatic hydrocarbon solvents from C6 to C17 aliphatic hydrocarbon solvents with substantially similar results as those described above.

While the processes according to the invention have been described as being carried out using particular temperature values and pressure values, it will be readily apparent to those skilled in the art that the process according to the invention may be carried out at other suitable temperature values and pressure values, and in general, the temperature values and pressure values at which the process according to the invention will be carried out, will be dependent on the solvent used.

It will also be appreciated that while the process according to the invention has been described as being carried out in a 1.2 litre soxhlet apparatus and in a particular type of commercial apparatus, the process according to the invention may be carried out in any other suitable apparatus, and when the process is carried out in a soxhlet apparatus, the volume of the soxhlet apparatus may be greater or less than 1.2 litres. It is also envisaged that the process according to the invention for solvent extraction of the oil extract from marine macro-algae may be carried out in a counter-flow continuous process whereby the desalted, dried and chopped pieces of marine macro-algae would be suitably contained to flow in one direction, and the solvent would be configured to flow in the opposite direction. In such a counter-flow system, it is envisaged that batches of the desalted dried and chopped pieces of marine macro-algae would be contained in mesh or other suitable porous containers and would be configured to flow in one direction, while the solvent would be configured to flow in the opposite direction.

While the solvent extraction process has been described whereby the solvent is recycled during the extraction process, in certain cases, it is envisaged that the solvent may not be recycled, and in which case, fresh solvent would be delivered to the marine macro-algae during the extraction process. The solvent could then be recovered when the oil extract is being recovered from the solvent.

1-98. (canceled)

99. A process for producing an oil extract from marine macro-algae, wherein the oil extract comprises dihomolinolenic acid, the process comprising extracting the oil from the marine macro-algae by solvent extraction, the solvent comprising one of an aliphatic hydrocarbon solvent from a C6 aliphatic hydrocarbon solvent to a C17 aliphatic hydrocarbon solvent.

100. A process as claimed in claim 99 in which the aliphatic hydrocarbon solvent comprises an aliphatic hydrocarbon solvent from a C6 aliphatic hydrocarbon solvent to a C15 aliphatic hydrocarbon solvent, and preferably the aliphatic hydrocarbon solvent comprises an aliphatic hydrocarbon solvent up to a C12 aliphatic hydrocarbon solvent, and advantageously, the aliphatic hydrocarbon solvent comprises an aliphatic hydrocarbon solvent up to a C10 aliphatic hydrocarbon solvent.

101. A process as claimed in claim 99 in which the solvent comprises a food grade solvent, and preferably, the solvent comprises hexane.

102. A process as claimed in claim 99 in which the dihomolinolenic acid constitutes not less than 7% by weight of the oil extract, and preferably, the oil extract constitutes dihomolinolenic acid in an amount not less than 8% by weight of the oil extract, and advantageously, the oil extract constitutes dihomolinolenic acid in an amount not less than 10% by weight of the oil extract, and preferably, the oil extract constitutes dihomolinolenic acid in an amount not less than 11% by weight of the oil extract, and advantageously, the oil extract constitutes dihomolinolenic acid in an amount not less than 12% by weight of the oil extract, and preferably, the dihomolinolenic acid constitutes in the range of 7% to 15% by weight of the oil extract, and preferably, the dihomolinolenic acid constitutes in the range of 8% to 15% by weight of the oil extract, and advantageously, the dihomolinolenic acid constitutes in the range of 10% to 15% by weight of the oil extract, and preferably, the dihomolinolenic acid constitutes in the range of 11% to 15% by weight of the oil extract.

103. A process as claimed in claim 99 in which the marine macro-algae comprises an intertidal marine macro-algae, and preferably, the marine macro-algae comprises marine macro-algae commonly found in the north east Atlantic Ocean, and advantageously, the marine macro-algae comprises one or more of the following species: ascophyllum; Fucus vesiculosus; pelvetia canaliculata; cystoseira tamariscifolia; cystoseira nodicaulis; porphyra dioica, and preferably, the marine macro-algae comprises ascophyllum.

104. A process as claimed in claim 99 in which the marine macro-algae is placed in a first vessel, and the solvent is delivered into the first vessel to extract the oil from the marine macro-algae, and preferably, the solvent is continuously delivered to the first vessel, and advantageously, the solvent with the oil extract entrained therein is continuously delivered from the first vessel, and preferably, the solvent is drip-fed into the first vessel, and advantageously, the first vessel comprises a pressurised vessel, and preferably, the solvent extraction is carried out in the first vessel under pressure, and preferably, the solvent extraction is carried out in the first vessel at a pressure in the range of 10 bar to 50 bar absolute pressure, and advantageously, the solvent extraction is carried out in the first vessel at a pressure of approximately 40 bar absolute pressure.

105. A process as claimed in claim 104 in which the solvent is supplied to the first vessel from a second vessel, and preferably, the first vessel comprises a process vessel of a soxhlet apparatus and the second vessel comprises a solvent reservoir attached to the soxhlet apparatus for storing the solvent and receiving the solvent with the oil extract entrained therein from the process vessel, and advantageously, the marine macro-algae is covered in the first vessel with a silica glass wool, and preferably, the marine macro-algae is packed into the first vessel.

106. A process as claimed in claim 105 in which the solvent with the oil extract entrained therein is returned from the first vessel to the second vessel, and preferably the solvent in the second vessel is evaporated and the solvent evaporate is delivered to the first vessel, and advantageously, the second vessel is heated to raise the temperature of the contents therein to the boiling point of the solvent, and preferably, the solvent evaporate is condensed prior to delivery to the first vessel, and advantageously, the marine macro-algae is retained in a porous container in the first vessel, and preferably, the porous container comprises a mesh container, or alternatively, the porous container comprises a cellulose container, and preferably, the oil extraction process is carried out in the first vessel at room temperature.

107. A process as claimed in claim 104 in which the oil extraction process is continued until the solvent being delivered from the first vessel contains substantially no oil, and preferably, the oil extraction process is continued until substantially all the oil being extracted from the marine macro-algae has been extracted therefrom, and advantageously substantially all the oil being extracted from the marine macro-algae is determined as having been extracted therefrom by inspecting the solvent being delivered from the first vessel, and preferably, substantially all the oil being extracted from the marine macro-algae is determined as having been extracted from the marine macro-algae by inspecting the colour of the solvent being delivered from the first the vessel, and advantageously, the oil extraction process is continued until the solvent being delivered from the first vessel is colourless.

108. A process as claimed in claim 99 in which the oil extract entrained in the solvent is recovered from the solvent by distillation, and preferably, solvent evaporated during distillation is condensed for subsequent use.

109. A process as claimed in claim 108 in which distillation is carried out at low pressure, and advantageously, distillation is carried out at a pressure in the range of 10 mbar to 100 mbar absolute pressure, and preferably, distillation is carried out at a pressure in the range of 20 mbar to 30 mbar absolute pressure, and advantageously, the distillation is carried out at a pressure of approximately 20 mbar absolute pressure, and preferably, the distillation is carried out at a temperature of the boiling point of the solvent corresponding to the pressure at which the distillation is being carried out, and advantageously, the distillation is carried out until the solvent contained in the oil extract does not constitute more than 5% by weight of the recovered oil extract.

110. A process as claimed in claim 108 in which the solvent with the oil extract entrained therein is cooled prior to distillation, and preferably, the solvent with the oil extract entrained therein is cooled to a temperature in the range of 20° C. to 30° C. prior to distillation, and advantageously the solvent with the oil extract entrained therein is cooled to a temperature of approximately of 25° C. prior to distillation.

111. A process as claimed in claim 108 in which the oil extract recovered from distillation is subjected to desolvating until substantially all the solvent has been removed from the recovered oil extract or the amount of solvent remaining in the recovered oil extract has been reduced to a negligible amount, and preferably, the desolvating of the recovered oil extract from distillation is carried out at a temperature in the range of 30° C. to 50° C., and advantageously, the desolvating of the recovered oil extract from distillation is carried out at a temperature in the range of 40° C. to 45° C., and preferably, the desolvating of the recovered oil extract from distillation is carried out at a temperature of approximately 41° C.

112. A process as claimed in claim 111 in which the desolvating of the recovered oil extract from distillation is carried out in a vacuum, and advantageously, the desolvating of the recovered oil extract from distillation is carried out at a pressure in the range of 10 mbar to 50 mbar absolute pressure, and preferably, the desolvating of the recovered oil extract from distillation is carried out at a pressure in the range of 20 mbar to 30 mbar absolute pressure, and advantageously, the desolvating of the recovered oil extract from distillation is carried out at a pressure of approximately 20 mbar absolute pressure.

113. A process as claimed in claim 111 in which the recovered oil extract from distillation is cooled prior to the desolvating thereof, and advantageously, the recovered oil extract from distillation is cooled to a temperature in the range of 20° C. to 30° C. prior to desolvating thereof, and preferably, the recovered oil extract from distillation is cooled to a temperature of approximately 25° C. prior to desolvating thereof.

114. A process as claimed in claim 99 in which the marine macro-algae is desalted prior to the solvent extraction process, and preferably, the marine macro-algae is desalted until the residual salt in the marine macro-algae does not exceed 7% by weight, and advantageously, the marine macro-algae is desalted until the residual salt in the marine macro-algae does not exceed 5% by weight, and preferably, the marine macro-algae is desalted until the residual salt in the marine macro-algae does not exceed 3% by weight.

115. A process as claimed in claim 99 in which the marine macro-algae is dried prior to the solvent extraction process, and preferably, the marine macro-algae is dried until the moisture content thereof is less than 10% by weight, and advantageously, the marine macro-algae is dried until the moisture content thereof is less than 8% by weight, and preferably, which the marine macro-algae is dried until the moisture content thereof is less than 5% by weight, and advantageously, the marine macro-algae is dried until the moisture content thereof is less than 3% by weight, and preferably, the marine macro-algae is dried subsequent to being desalted.

116. A process as claimed in claim 99 in which the marine macro-algae is chopped into pieces prior to the solvent extraction process, and preferably, the marine macro-algae is chopped into pieces of size, the maximum dimension of each piece not exceeding 10 mm, and advantageously, the marine macro-algae is chopped into pieces of size, the maximum dimension of each piece lying in the range of 100 microns to 10 mm, and preferably, the marine macro-algae is chopped into pieces of size, the maximum dimension of each piece not exceeding 8 mm, and advantageously, the marine macro-algae is chopped into pieces of size, the maximum dimension of each piece not exceeding 5 mm, and preferably, the marine macro-algae is chopped into pieces of size, the maximum dimension of each piece lying approximately 4 mm, and advantageously, the marine macro-algae is chopped into pieces subsequent to being dried.

117. A process as claimed in claim 99 in which the oil extract is microencapsulated.

118. An oil extract extracted from marine macro-algae by a process as claimed in claim 99, wherein the oil extract comprises dihomolinolenic acid.
